(19) United States
(12) Patent Application Publication
(10) Pub. No.: US 2014/0155940 A1
(43) Pub. Date: Jun. 5, 2014

(54) INTERVERTEBRAL DYNAMIC FIXATION IMPLANT

(71) Applicants: Gilles Dubois, Toulouse (FR); Jacques Senegas, Merignac (FR)

(72) Inventors: Gilles Dubois, Toulouse (FR); Jacques Senegas, Merignac (FR)

(21) Appl. No.: 14/236,035

(22) PCT Filed: Jul. 26, 2012
(86) PCT No.: PCT/FR2012/051780

§ 371 Date: Jan. 29, 2014

(30) Foreign Application Priority Data
Aug. 2, 2011 (FR) 1157095

(51) Int. Cl. A61B 17/70

(52) U.S. Cl. CPC A61B 17/7067
USPC 606/249

(57) ABSTRACT

The invention concerns an intervertebral dynamic fixation implant intended for stabilization of the lumbar spine in one or more intervertebral segments, comprising: an intervertebral wedge extending along a vertical axis, comprising two craniocaudal notches intended for receiving the spinous processes of two lumbar vertebrae Ln and Ln+1, a vertical channel being arranged on the two lateral faces of said intervertebral wedge, a rigid strip covering a portion of the back face and of the two lateral faces of the intervertebral wedge, so as to leave a passageway in the vertical side channels, at least one hole passing through the intervertebral wedge from one lateral face to the other, in the caudal portion of the rigid strip, two pedicle screws fixed to the lumbar vertebra Ln+1, comprising a system for tensioning and attaching a braid Tn which wraps around the spinous process of lumbar vertebra Ln and passes through the passageways of the side channels of the intervertebral wedge, a rod perpendicular to the vertical axis of the intervertebral wedge, connecting the two pedicle screws by passing through said at least one hole located in the caudal portion of the rigid strip.

### BACKGROUND

1. Technical Field

The present invention relates to the field of spinal implants. It concerns an implant for retaining vertebral bodies while allowing a sufficient degree of freedom to permit movement of the spine in all three planes.

2. Description of the Related Art

In the musculoskeletal system, the spine acts to stabilize the trunk and to support the pectoral and pelvic girdles. These functions can be altered by natural wear, pathological degeneration, or traumatic shock, resulting in discomfort, pain, or even severe disability in subjects suffering from these disorders.

The effectiveness of the load distribution and the load balancing, both static and mobile, are determined by coordinating the various component segments of the spine, divided into functional units. Each functional unit consists of two adjacent vertebrae connected by an intervertebral disc. Stacked atop one another, they form the spinal joint complex.

Various surgical techniques have been proposed for treating the affected spinal joint complex, such as bone fusion (arthrodesis) or instrumented stabilization of vertebral bodies.

Although it is accompanied by a loss of mobility in the section of the spinal column where the surgery is performed, fusion of vertebral bodies is a longstanding practice because of its rapid and effective results. However, studies conducted on such patients five to ten years after surgery have showed that it causes overloading at the junction of the operated area, with the appearance of herniated discs, osteosclerosis, and persistent pain.

To reduce these drawbacks, the use of implants with dynamic attachment to the vertebral body was invented for certain indications. The purpose of these implants is to provide a flexible system, using connectors or spacers, fastened with screws placed in the vertebral pedicles, to achieve dynamic stabilization while avoiding fusion of the vertebral bodies. These implants are specifically intended for treatment of the lumbosacral spine. An example of this type of device is represented by the Dynesys® implant (Zimmer).

Such implants typically include a thermoplastic cylindrical spacer attached by its two ends to two adjacent vertebral bodies by means of conical transpedicular screws, and traversed by a tensioning cord acting as an artificial ligament. The retention of a functional unit by this type of device involves attaching two spacers, one on each side of the vertebral centerline, using two pedicle screws each for a total of four screws.

This system provides stabilization of the spine with good retention of the vertebral bodies. Immobilization of the functional unit is reduced compared to spinal fusion techniques, allowing the patient to perform certain movements, particularly flexion/extension in the sagittal plane, which results in better post-operative recovery through suitable rehabilitation exercises. The patient can achieve spinal movements of greater amplitude compared to arthrodesis, even if ultimately their mobility remains relatively limited, partly because of the restrictive anchoring resulting from the attachment of four pedicle screws per functional unit. Other instrumental stabilization devices have been devised to eliminate the use of pedicle screws. An example of this type of device is represented by the Wallis® implant (Zimmer). Here, a spacer is inserted between two vertebral bodies, held in place by a flexible strap wrapping around the two spinous processes of adjacent vertebrae and connected to end fasteners which allow adjusting the length of the flexible strap and which are attached to the main body of the spacer by snapping them in place.

This system also enhances the mobility of a functional unit treated in this manner, compared to previous techniques. It has the advantage of allowing minimally invasive and reversible surgery, as the implant can be easily removed without causing bone lesions. However, the attachment system to the spinous processes has a certain laxity, as the tension of the flexible strap tends to decrease with spinal movements, which leads to a reduction in the efficiency of the implant over time.

### BRIEF SUMMARY

To overcome the drawbacks of the prior art, the present invention proposes a new implant for dynamic fixation of vertebral bodies, without bone fusion, providing good vertebral anchorage, so as to ensure an effective and lasting stabilization over time of one or more functional units while allowing increased mobility of the spine.

One objective of the spinal implant of the invention is to limit the traumatic shock related to surgery, in particular by reducing the number of pedicle screws used during the surgery.

Another objective of the spinal implant of the present invention is to provide a robust and intimate fixation system for attachment to the vertebral bodies, easily readjustable to the anatomy of the patient during the operation.

Another objective of the spinal implant of the present invention is to provide stabilization of the vertebral body with good rigidity, while allowing mobility of the spine in the three planes: the coronal plane for lateral abduction and adduction movements, the sagittal plane for flexion/extension movements, and the axial plane for rotational movements of the vertebrae relative to each other.

These objectives are achieved by means of the present invention, which relates more specifically to an intervertebral dynamic fixation implant for stabilization of the lumbar spine in one or more vertebral segments, comprising:
an intervertebral wedge extending along a vertical axis, comprising two craniocaudal notches for receiving the spinous processes of two lumbar vertebrae Ln and Ln+1, a vertical channel being arranged on the two lateral faces of said intervertebral wedge,
a rigid strip covering a portion of the back face and the two lateral faces of the intervertebral wedge, so as to leave a passageway in the vertical side channels,
at least one hole passing through the intervertebral wedge from one lateral face to the other, in the caudal portion of the rigid strip,
two pedicle screws fixed to the lumbar vertebra Ln+1, comprising a system for tensioning and attaching a braid Tn wrapping the spinous process of the lumbar vertebra Ln and passing through the passageways of the side channels of the intervertebral wedge,
a rod perpendicular to the vertical axis of the intervertebral wedge, connecting the two pedicle screws by passing through said at least one hole located in the caudal portion of the rigid strip.

The intervertebral wedge according to the invention has a configuration allowing its complete insertion between the two vertebral bodies to be treated. For this purpose, the cranial-caudal notches snugly fit against the spinous processes of the vertebrae between which the intervertebral wedge is placed. A degree of freedom exists, however, between the two spinous processes because of the dynamic properties of the intervertebral wedge, which is composed of a material having a certain elasticity. Flexion/extension of the column in the sagittal plane are thus possible, the two spinous processes of the same functional unit being able to draw closer together or move further apart. As its dimensions will vary according to the anatomy of the patient, the length of the intervertebral wedge of the invention is determined by the surgeon, based on the surgery. As an indication, the intervertebral wedge may have a length of between about ten and thirty millimeters, and a width of about ten millimeters.

Several compounds can be chosen to create the intervertebral wedge of the invention. The chosen material must have a certain elasticity to allow slight deformations of the intervertebral wedge during spinal movements, including flexion/extension. Those skilled in the art will know how to select the proper materials for the case at hand and for the desired rigidity. Plastics and associated materials such as polycarbonate urethane or silicone can be chosen. Preferably, the intervertebral wedge is advantageously composed PEEK (polyether ether ketone).

The lateral faces of the intervertebral wedge according to the invention have a slight recess cut into them, forming a channel extending along a vertical craniocaudal axis on each side of said two lateral faces. Measuring about one to two millimeters in depth, this recess can receive the braid in contact with the lateral faces of the intervertebral wedge so that the channel this forms can guide the braid.

The rigid strip of the invention covers a portion of the back face of the intervertebral wedge and both lateral faces of this wedge, so that it passes over the vertical channel formed in the lateral faces, leaving a passageway through which the braid can run. In one particular embodiment of the invention, the rigid strip is U-shaped, measuring between approximately forty and sixty millimeters in total length, ten to twelve millimeters in width, and 0.8 to one millimeter thick.

The attachment of the rigid strip to the intervertebral wedge can be achieved in various ways, for example by clipping it in place or gluing it. Advantageously, the rigid strip is attached to the back face of the intervertebral wedge by a screw. The composition of the rigid strip may also be variable, as long as it can fulfill its role of covering the intervertebral wedge and particularly the vertical channels present on the lateral faces. Preferably, the rigid strip is metal.

The lateral faces of the intervertebral wedge according to the invention have at least one hole in each of said faces, in the caudal portion relative to the rigid strip covering the channel in these faces. Preferably, this hole is located in the vertical channel of the lateral faces, on an axis perpendicular to the center axis of the implant according to the invention. Said at least one hole may have a variable number of shapes. Its function is primarily to allow the passage of the rod perpendicular to the vertical axis of the intervertebral wedge.

In one particular embodiment of the invention, said at least one hole also allows the passage of a braid Tn+1 wrapped around the spinous process of a lumbar vertebra Ln+1 connected to another intervertebral dynamic fixation implant which is also in contact with a lumbar vertebra Ln +2.

Alternatively, said at least one hole according to the invention allows the passage of the braid and of the rod independently, meaning through two compartments located within said at least one hole and separated by a partition.

One of the decisive advantages of the implant according to the invention is that it enables the stabilization of a functional unit by fixation with only two pedicle screws, distributed in each of the vertebral pedicles of the same vertebra. Contrary to the prior art, the two vertebrae Ln and Ln+1 are part of the same functional unit and are held in place by the implant of the invention by attaching screws in the vertebral pedicles of vertebra Ln+1, instead of the four screws distributed among the vertebral pedicles of vertebrae Ln and Ln+1 of the prior art devices.

The pedicle screws used according to the invention have a system for tensioning and securing a braid Tn wrapped around the spinous process of vertebra Ln. In one particular embodiment of the invention, the heads of the pedicle screws have a space adapted to receive a portion of the braid, and by a tightening mechanism well known to those skilled in the art, they allow fixation by immobilization of said braid after the surgeon has determined the length of braid necessary for proper fixation of the implant of the invention.

The braid of the invention is thus attached by its two ends to the two pedicle screws. The body of the braid runs along the periphery of the implant of the invention, in contact with the lateral faces, inside vertical channels formed in these faces and covered by the rigid strip. The braid allows fitting the cranial notch closely to the spinous process of the vertebra Ln by the tensioning applied by the surgeon during the operation when he adjusts the tightening mechanism of the two pedicle screws.

The nature and form of the braid of the invention may vary, as long as it offers sufficient flexibility and strength to perform its function. It can be of plastic derivatives such as polyethylene terephthalate. Preferably, the braid according to the invention is of polyester and has a flattened shape that encourages its grip on the implant and on the spinous process.

The fixation of the implant according to the invention is completed by a rod perpendicular to the vertical axis of the intervertebral wedge, interconnecting the two pedicle screws by passing through said at least one hole traversing the intervertebral wedge from one lateral face to the other in the caudal portion of the rigid strip. This fixation helps reinforce the grip of the implant on vertebra Ln+1 and consolidates the stability of the functional unit. This original fixation system ensures complete retention of the intervertebral wedge, which is already tied to the spinous process of vertebra Ln by the braid Tn. It also limits the corollary lateral “bending” phenomena of vertebral instabilities in the rotational movements of the vertebrae.

The rod used for this fixation must be sufficiently strong. It can be of materials derived from plastic such as PEEK or other sufficiently resistant materials. Advantageously, the rod according to the invention is made of metal, such as titanium.

### BRIEF DESCRIPTION OF THE SEVERAL VIEWS OF THE DRAWINGS

The following examples and illustrate the invention in order to highlight the features and benefits more clearly, although this does not reduce the scope in any way.

FIG. 1: schematic representation of the back face of an intervertebral wedge according to the invention,

FIG. 2: schematic representation of the back face of a rigid strip according to the invention,

FIG. 3: schematic representation of the back face of an intervertebral wedge covered by a rigid strip according to the invention,

FIG. 4: schematic representation of a side view of an intervertebral wedge covered by a rigid strip according to the invention,

FIG. 5: schematic representation of the back face of two implants according to the invention, attached to two functional units comprising three vertebrae Ln, Ln+1 and Ln+2,

FIG. 6: schematic representation of a side view of two implants according to the invention, attached to two functional units comprising three vertebrae Ln, Ln+1 and Ln+2.

### DETAILED DESCRIPTION

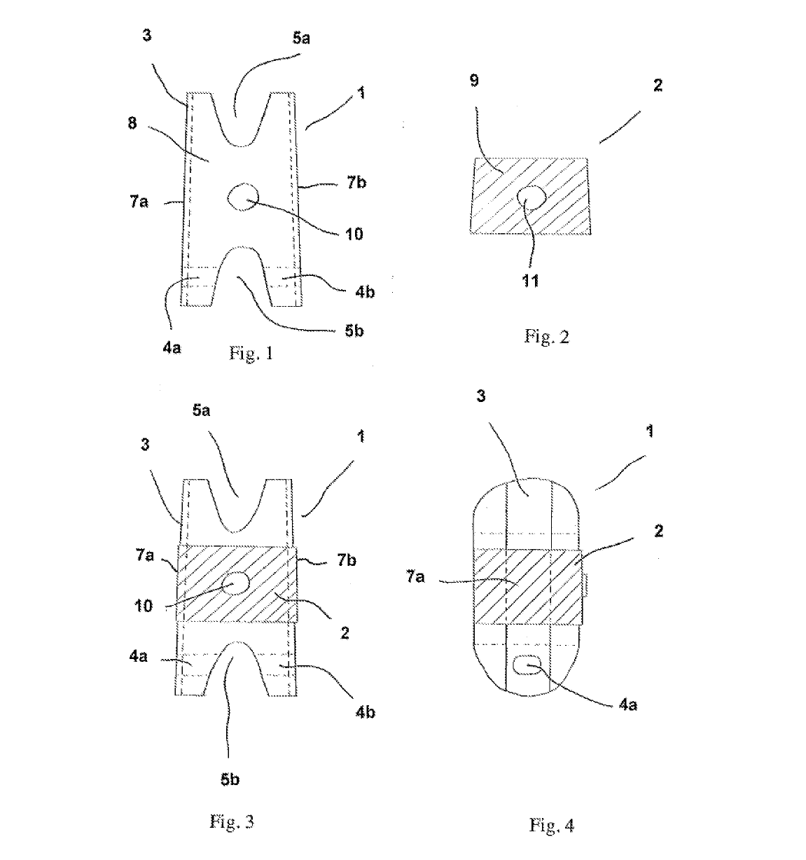

FIG. 1 shows an example of an intervertebral wedge 1 according to the invention, in a view of its back face 8. Shaped as a slightly conical X with a cranial width substantially less than its caudal width, it has two notches 5a and 5b in the cranial and caudal portions respectively. A hole 10 is provided at the center of the wedge to allow attaching the rigid strip 2. The material chosen in this example is PEEK.

FIG. 2 shows an example of a rigid strip 2 according to the invention, in a view of its back face 9. Having a shape complementary to the intervertebral wedge 1, it partly covers the back face 8 of the latter as well as part of the two lateral faces 7a and 7b. It also has a hole 11, positioned so as be superimposed over the hole 10 present in the intervertebral wedge 1 to allow screw attachment of the rigid strip 2. The material chosen in this example is a biocompatible metal.

FIG. 3 shows the intervertebral wedge 1 covered by the rigid strip 2 according to the invention. Note that the rigid strip 2 covers the back face 8 of the intervertebral wedge 1 without overlapping the notches 5a and 5b, so as not to interfere with their insertion against the spinous processes of the vertebrae Ln and Ln+1 to which they are to be attached.

FIG. 4 is a profile view of the intervertebral wedge 1 covered by the rigid strip 2, as shown in FIG. 3. In this example, the profile of the intervertebral wedge 1 is slightly rounded at its ends to provide a better fit against the spinous processes. The left lateral face 7a is visible, with the vertical channel 3 present on this face partially exposed, extending from the caudal end to the cranial end along the vertical axis of the intervertebral wedge 1. The hole 4a located in the caudal portion of the rigid strip 2, within the vertical channel 3, can also be seen in this view. It is in alignment with the hole 4b located in the right lateral portion of the intervertebral wedge 1, to allow receiving the rod 12.

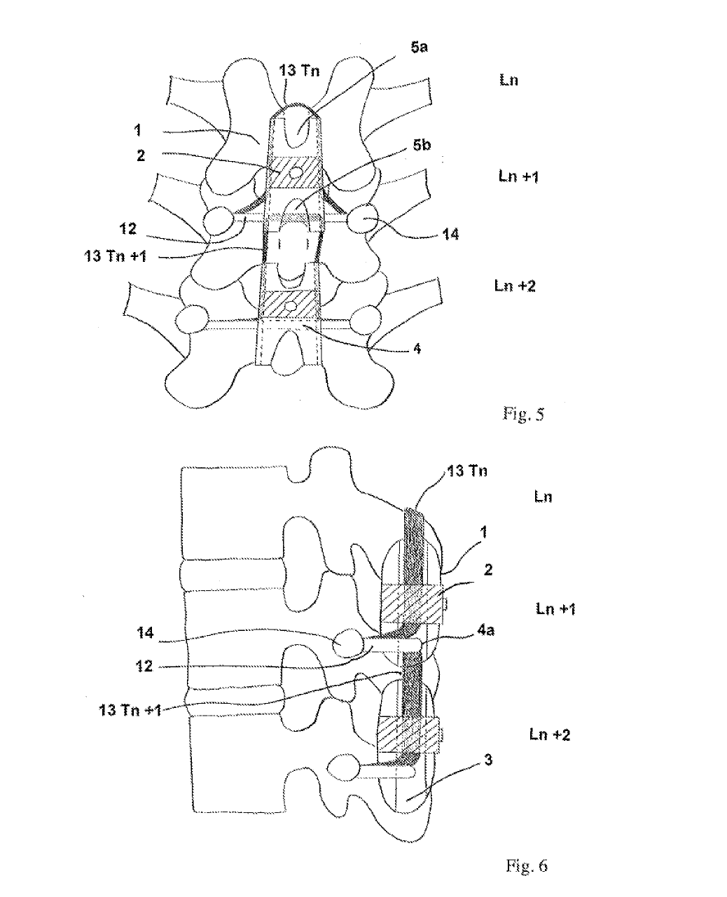

FIG. 5 shows two implants according to the invention, in a view of their back faces, fixed to two functional units comprising three lumbar vertebrae designated Ln, Ln+2, and Ln+1. A first implant is fixed between the spinous processes of vertebrae Ln and Ln+1 and a second implant is fixed between the spinous processes of vertebrae Ln+1 and Ln+2.

The cranial 5a and caudal 5b notches of the first implant are inserted respectively around the spinous processes of vertebrae Ln and Ln+1. A braid 13 Tn of polyester keeps the notch 5a in close contact with the spinous process of vertebra Ln by wrapping around it. The braid then runs along the lateral faces 7a and 7b of the intervertebral wedge 1 of the first implant, passing through the vertical channel 3 where it is covered by the rigid strip 2 which keeps the braid 13 Tn within the channel 3. The two ends of the braid Tn 13 are secured to the pedicle screws 14 implanted in the pedicles of vertebra Ln+1. The attachment of the braid 13 Tn to the pedicle screws Tn is done by the surgeon during the operation, by a tightening system present on the heads of the pedicle screws 14, which tensions the braid 13 Tn so that it is firmly wrapped around the spinous process of the vertebra Ln to which the intervertebral wedge 1 is anchored.

The perpendicular rod 12 is also attached by its two ends to the heads of the pedicle screws 14. The view in FIG. 5 clearly shows that the first implant can stabilize a first functional unit comprising vertebrae Ln and Ln+1 by the placement of only two pedicle screws in vertebra Ln+1. The vertebra Ln is securely tied to the intervertebral wedge 1 by the braid 13 Tn, securing the cranial notch 5a to the spinous process of this vertebra. The attachment of the intervertebral wedge 1 of the first implant is completed by immobilizing the ends of the braid 13 Tn at the pedicle screws 14 located in vertebra Ln+1. This attachment is further reinforced by the perpendicular rod 12, also immobilized by the pedicle screws 14, passing through the holes 4a and 4b located on the lateral faces 7a and 7b of the intervertebral wedge 1.

The intervertebral wedge 1 of the first implant is therefore firmly anchored to vertebrae Ln and Ln+1 by several points of attachment, allowing it to fully fulfill its role of stabilizing the functional unit. This stabilization is further achieved by the placement of only two pedicle screws, which reduces the traumatic shock associated with this operation and also frees some of the space surrounding the treated vertebrae, facilitating the movement of this vertebral joint in all three planes.

A second implant of the invention is also shown in FIG. 5, attached between vertebrae Ln+1 and Ln+2 of a second functional unit adjacent to the first. In this example, the notch 5a of the second implant is placed in close contact with the spinous process of vertebra Ln+1 by means of the braid 13 Tn. This braid may directly wrap around the spinous process of vertebra Ln+1 in the same manner that the braid Tn of the first implant wraps around the spinous process of vertebra Ln. FIG. 5 illustrates another method, however, for connecting the second implant to the first implant by running the braid 13 Tn not directly around the spinous process of vertebra Ln+1, but indirectly through the holes 4a and 4b of the first implant. This achieves a synergistic action between the two implants, contributing to better stabilization of the two functional units so treated, by reinforcing the anchoring of the first implant to the spinous process of vertebra Ln+1, and by providing a solid connection between the first implant and the second implant.

FIG. 6 provides a side view more clearly distinguishing the connection between the two implants according to the invention. In this example, the braid 13 Tn+1 passes through the same hole 4a as the perpendicular rod 12 of the first implant, although, in accordance with the invention, it could pass through a separate compartment located in the same area as the hole 4a.

One will note that the stabilization of two functional units comprising three vertebrae Ln, Ln+1, and Ln+2 by two implants according to the invention only requires the placement of two pedicle screws per vertebrae Ln+1 and Ln+2, one screw on each side for a total of four screws. This savings does not weaken the attachment of the implants to the vertebrae nor their stabilization effect, but does improve the mobility of the two functional units so treated.

1. An intervertebral dynamic fixation implant intended for stabilization of the lumbar spine in one or more vertebral segments, comprising:
an intervertebral wedge extending along a vertical axis, the intervertebral wedge comprising;
two craniocaudal notches for receiving spinous processes of two lumbar vertebrae,
first and second lateral faces, and
first and second vertical channels arranged respectively on the first and second lateral faces of said intervertebral wedge,
a rigid strip covering a portion of a back face and respective portions of the first second lateral faces of the intervertebral wedge, so as to leave respective passageways in the vertical channels,
at least one hole passing through the intervertebral wedge from the first lateral face to the second lateral face, in a caudal portion of the rigid strip,
a braid configured to wrap the spinous process of the two lumbar vertebra and passing through the passageways of the vertical channels of the interbertebral wedge,
two pedicle screws configured to be fixed to the two lumbar vertebra, the pedicle screws comprising a system for tensioning and attaching to the braid,
a rod perpendicular to the vertical axis of the intervertebral wedge, connecting the two pedicle screws by passing through said at least one hole located in the caudal portion of the rigid strip.

2. The intervertebral dynamic fixation implant according to claim 1, wherein said at least one hole comprises two compartments separated by a partition, allowing the passage of a braid, wrapped around the spinous process of a lumbar vertebra hi -connected to another intervertebral dynamic fixation implant that is independently also in contact with a lumbar vertebra, and of the rod.

3. The intervertebral dynamic fixation implant according to claim 1, wherein the intervertebral wedge is polyether ether ketone (PEEK).

4. The intervertebral dynamic fixation implant according to claim 1, wherein the rigid strip is attached to the back face of the intervertebral wedge by a screw.

5. The intervertebral dynamic fixation implant according to claim 1, wherein the rigid strip is metal.

6. The intervertebral dynamic fixation implant according to claim 1, wherein the braid is polyester and has a flattened shape.

7. The intervertebral dynamic fixation implant according to claim 1, wherein the rod is metal.
